(19) United States
(12) Patent Application Publication
(10) Pub. No.: US 2023/0212342 A1
(43) Pub. Date: Jul. 6, 2023

(54) RESIN COMPOSITION AND RESIN FILM

(71) Applicant: INDUSTRIAL TECHNOLOGY RESEARCH INSTITUTE, Hsinchu (TW)

(72) Inventors: Yi-Chun CHEN, Hsinchu City (TW); Yu-Chen KAO, Hsinchu County (TW); Kai-Chi CHEN, Zhubei City (TW)

(73) Assignee: INDUSTRIAL TECHNOLOGY RESEARCH INSTITUTE, Hsinchu (TW)

(21) Appl. No.: 17/897,288

(22) Filed: Aug. 29, 2022

(30) Foreign Application Priority Data
Dec. 30, 2021 (TW) 110149642

(51) Int. Cl. C08F 283/10

(52) U.S. Cl. CPC C08F 283/10

(57) ABSTRACT

A resin composition and a resin film are provided. The resin composition includes a hardener and an epoxy resin monomer. The epoxy resin monomer has a structure represented by Formula (I)

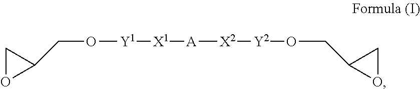

wherein A is substituted or unsubstituted C₆₋₂₄ arylene group, C₃₋₁₆ cycloalkylene group, C₃₋₁₆ heteroarylene group, C₃₋₁₆ alicyclic alkylene group, or divalent C₆₋₂₅ alkylaryl group; X¹ and X² are independently

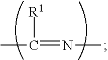

Y¹ and Y² are independently substituted or unsubstituted C₆₋₂₄ arylene group, and Y¹ is different from Y²; and R¹ is hydrogen, C₁₋₈ alkyl group, or C₁₋₈ alkoxy group, wherein the weight ratio of the curing agent to the epoxy resin monomer having a structure represented by Formula (I) is from 1:100 to 1:1.

### CROSS REFERENCE TO RELATED APPLICATIONS

The application is based on, and claims priority from, Taiwan Application Serial Number 110149642, filed on Dec. 30, 2021, the disclosure of which is hereby incorporated by reference herein in its entirety.

### TECHNICAL FIELD

The disclosure relates to a resin composition and a resin film.

### BACKGROUND

Anisotropic conductive adhesive is widely used in printed contacts to connect indium tin oxide (ITO) substrates with driver circuit substrates (such as soft printed circuit boards). Due to their excellent mechanical properties, chemical resistance, thermal tolerance and insulation properties, thermosetting resins are generally used in mainstream anisotropic conductive adhesives.

However, since a thermosetting resin cannot be melted nor dissolved after curing, it is difficult to reshape and reprocess, or to recycle a product made of thermosetting resin. Therefore, the use of anisotropic conductive adhesive employing a conventional thermosetting resin leads to problems such as difficulties with disassembly and removal of residual adhesive. In addition, in the field of electronic materials, new semiconductor packaging is gradually being developed that is smaller, thinner, and having a more complex shape. Therefore, with the increasing difficulties with packaging, the industry's demand for semiconductor packaging materials with high flowability and low stress is also increasing.

### SUMMARY

According to embodiments of the disclosure, the disclosure provides a resin composition such as a thermosetting resin composition. The resin composition of the disclosure includes a hardener; and an epoxy resin monomer. The epoxy resin monomer has a structure represented by Formula (I)

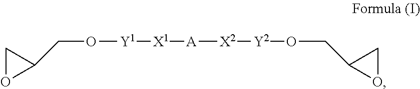

wherein A can be substituted or non-substituted C₆₋₂₄ arylene group, substituted or non-substituted C₃₋₁₆ cycloalkylene group, substituted or unsubstituted C₃₋₁₆ heteroarylene group, substituted or non-substituted C₃₋₁₆ alicyclic alkylene group, or substituted or non-substituted divalent C₆₋₂₅ alkylaryl group; X¹ and X² can be independently

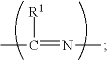

Y¹ and Y² can be independently substituted or non-substituted C₆₋₂₄ arylene group, and Y¹ is distinct from Y²; and, R¹ can be hydrogen, C₁₋₈ alkyl group, or C₁₋₈ alkoxy group. According to embodiments of the disclosure, the weight ratio of the hardener to the epoxy resin monomer can be 1:100 to 1:1.

According to embodiments of the disclosure, the resin composition may additionally further includes an epoxy resin. According to embodiments of the disclosure, the weight ratio of the epoxy resin to the epoxy resin monomer having a structure represented by Formula (I) can be 1:100 to 9:1.

According to some embodiments of the disclosure, the disclosure provides a resin film. The resin film can include the cured product of the resin composition of the disclosure.

A detailed description is given in the following embodiments.

### DETAILED DESCRIPTION

The resin composition and resin film are described in detail in the following description. In the following detailed description, for purposes of explanation, numerous specific details and embodiments are set forth in order to provide a thorough understanding of the present disclosure. The specific elements and configurations described in the following detailed description are set forth in order to clearly describe the present disclosure. It will be apparent, however, that the exemplary embodiments set forth herein are used merely for the purpose of illustration, and the inventive concept may be embodied in various forms without being limited to those exemplary embodiments. As used herein, the term “about” in quantitative terms refers to plus or minus an amount that is general and reasonable to persons skilled in the art.

The disclosure provides a resin composition and a resin film. According to embodiments of the disclosure, the resin film can be a film prepared by curing the resin composition. According to embodiments of the disclosure, the resin composition includes an epoxy resin monomer having a specific structure and a hardener. Since an imine moiety is introduced into the epoxy resin monomer of the disclosure, the cured product of the resin composition of the disclosure can be decomposed under an acidic condition at a relatively low temperature (e.g., equal to or less than 80° C.), thereby solving the problems of thermosetting resin that does not decompose easily. As a result, when the resin composition of the disclosure serves as a packaging adhesive, the cured product of the resin composition can be easily removed under specific conditions. As a result, the adhesive residue problem can be avoided, and the device encapsulated by the resin composition can be easily disassembled and recycled. In addition, since the epoxy resin monomer of the disclosure has an aryl moiety, the device employing the resin composition of the disclosure (i.e. the device includes the cured product of the resin composition) exhibits low-temperature decomposability, high thermal tolerance, high chemical resistance, and dimensional stability. Furthermore, due to the asymmetrical chemical structure of the epoxy resin monomer, the melting point of the epoxy resin monomer of the disclosure can be greatly reduced (solving the problem of high melting point of conditional epoxy resin) such that the epoxy resin monomer of the disclosure is liquid at room temperature. As a result, due to the addition of the liquid epoxy resin monomer, the flowability of the resin composition of the disclosure can be greatly improved, thereby expanding the application scope of resin composition (e.g., package systems that require low-temperature operation or higher complexity). According to embodiments of the disclosure, the resin composition of the disclosure can serve as an adhesive for use in anisotropic conductive adhesive, underfill adhesive, b-stage adhesive film or liquid adhesive.

The resin composition of the disclosure includes a hardener, and an epoxy resin monomer. According to embodiments of the disclosure, the amount of the hardener is not limited and can be optionally modified by a person of ordinary skill in the field. According to embodiments of the disclosure, the weight ratio of the hardener to the epoxy resin monomer can be about 1:100 to 1:1, such as about 2:100, 3:100, 5:100, 8:100, 10:100, 15:100, 20:100, 25:100, 30:100, 40:100, 50:100, 60:100, 75:100, 80:100, or 90:100. According to embodiments of the disclosure, the epoxy resin monomer of the disclosure is suitable for use in concert with various hardener. The hardener is not limited and can be optionally selected by a person of ordinary skill in the field. According to embodiments of the disclosure, the hardener of the disclosure can be anhydride hardener, amine hardener, phenolic hardener, imidazole hardener, or a combination thereof. For example, the anhydride hardener can be methyl hexahydrophthalic anhydride, methyl tetrahydrophthalic anhydride (MTHPA), maleic anhydride (MA), polystyrene-co-maleic anhydride (SMA), or a combination thereof, but not limited to those described above. According to embodiments of the disclosure, the amine hardener can be aliphatic amine hardener, cycloaliphatic amine hardener, or aromatic amine hardener. The amine hardener can be poly(propylene glycol) bis(2-aminopropyl ether) (such as JEFFAMINE® D-230), cyclohexanediamine, oxydianiline, or stearyl amine ethoxylate (SAA). According to embodiments of the disclosure, phenolic hardener can be phenol-formaldehyde novolac (HRJ series), or melamine phenol novolac. According to embodiments of the disclosure, the imidazole hardener can be 1-methyl imidazole, 2-methyl imidazole, 2-ethyl-4-methyl imidiazole, 2-phenyl-4-methyl imidazole, 2-phenyl-4-methyl-5-hydroxymethyl imidazole, 2-phenyl-4,5-0, 2,4-diamino-6-[2′-methylimidazolyl-(1)]-ethyl-S-triazine, 2,4-diamino-6-(2′-undecyl imidazolyl)-ethyl-S-triazine, 2,4-diamino-6-[2′-ethyl-4-methyl imidazolyl-(1′)]-ethyl-S-triazine, or latent hardener (such as NOVACURE HXA-3932). The above hardeners are used as examples, and the anhydride hardener, amine hardener, phenolic hardener, and imidazole hardener should not be limited in the disclosure.

According to embodiments of the disclosure, the epoxy resin monomer of the disclosure can be an asymmetrical epoxy resin monomer (i.e. an epoxy resin monomer that has an asymmetrical chemical structure) with an intramolecular imino moiety. According to embodiments of the disclosure, the epoxy resin monomer of the disclosure can have a structure represented by Formula (I)

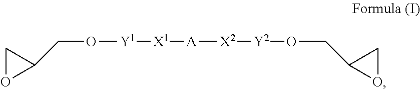

wherein A can be substituted or non-substituted C₆₋₂₄ arylene group, substituted or non-substituted C₃₋₁₆ cycloalkylene group, substituted or non-substituted C₃₋₁₆ heteroarylene group, substituted or non-substituted C₃₋₁₆ alicyclic alkylene group, or substituted or non-substituted divalent C₆₋₂₅ alkylaryl group; X¹ and X² can be independently

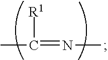

Y¹ and Y² can be independently substituted or non-substituted C₆₋₂₄ arylene group; and, R¹ can be hydrogen, C₁₋₈ alkyl group, or C₁₋₈ alkoxy group. It should be noted that, since the epoxy resin monomer of the disclosure is an asymmetrical epoxy resin monomer, Y¹ is distinct from Y². According to embodiments of the disclosure, Y¹ and Y² can be constructed by the same elements, but have different chemical structure.

According to embodiments of the disclosure, the substituted C₆₋₂₄ arylene group of the disclosure is a C₆₋₂₄ arylene group in which at least one hydrogen bonded to the carbon of the arylene group is replaced with a C₁₋₈ alkyl group, or C₁₋₈ alkoxy group; the substituted C₃₋₁₆ cycloalkylene group of the disclosure is a C₃₋₁₆ cycloalkylene group in which at least one hydrogen bonded to the carbon of the cycloalkylene group is replaced with a C₁₋₈ alkyl group, or C₁₋₈ alkoxy group; the substituted C₃₋₁₆ heteroarylene group of the disclosure is a C₃₋₁₆ heteroarylene group in which at least one hydrogen bonded to the carbon of the heteroarylene group is replaced with a C₁₋₈ alkyl group, or C₁₋₈ alkoxy group; the substituted C₃₋₁₆ alicyclic alkylene group of the disclosure is a C₃₋₁₆ alicyclic alkylene group in which at least one hydrogen bonded to the carbon of the alicyclic alkylene group is replaced with a C₁₋₈ alkyl group, or C₁₋₈ alkoxy group; and, the substituted divalent C₆₋₂₅ alkylaryl group of the disclosure is a divalent C₆₋₂₅ alkyl group in which at least one hydrogen bonded to the carbon of the divalent alkyl group is replaced with C₁₋₈ alkyl group, or C₁₋₈ alkoxy group.

According to embodiments of the disclosure, A can be substituted or non-substituted phenylene group, substituted or non-substituted biphenylene group, substituted or non-substituted naphthylene group, substituted or non-substituted thienylene group, substituted or non-substituted indolylene, substituted or non-substituted phenanthrenylene, substituted or non-substituted indenylene, substituted or non-substituted anthracenylene, or substituted or non-substituted fluorenylene, wherein the substituted phenylene group, substituted biphenylene group, substituted naphthylene group, substituted thienylene group, substituted indolylene, substituted phenanthrenylene, substituted indenylene, substituted anthracenylene, or substituted fluorenylene means such groups which at least one hydrogen bonded to the carbon thereof is replaced with a C₁₋₈ alkyl group or C₁₋₈ alkoxy group.

According to embodiments of the disclosure, C₁₋₈ alkyl group can be linear or branched alkyl group. For example, C₁₋₈ alkyl group can be methyl, ethyl, propyl, butyl, pentyl, hexyl, heptyl, octyl, or an isomer thereof. According to embodiments of the disclosure, C₁₋₈ alkyl group can be linear or branched alkoxy group. For example, C₁₋₈ alkoxy group can be methoxy, ethoxy, propoxy, butoxy, pentoxy, hexoxy, heptoxy, octoxy or an isomer thereof.

According to embodiments of the disclosure, X¹ can be bonded with Y¹ via a carbon atom on one side and bonded with A via a nitrogen atom on the other side, and X² is bonded with Y² via a carbon atom on one side and bonded with A via a nitrogen atom on the other side. In addition, according to embodiments of the disclosure, X¹ can be bonded with Y¹ via a nitrogen atom on one side and bonded with A via a carbon atom on the other side, and X² can be bonded with Y² via a nitrogen atom on one side and bonded with A via a carbon atom on the other side.

According to embodiments of the disclosure, A can be

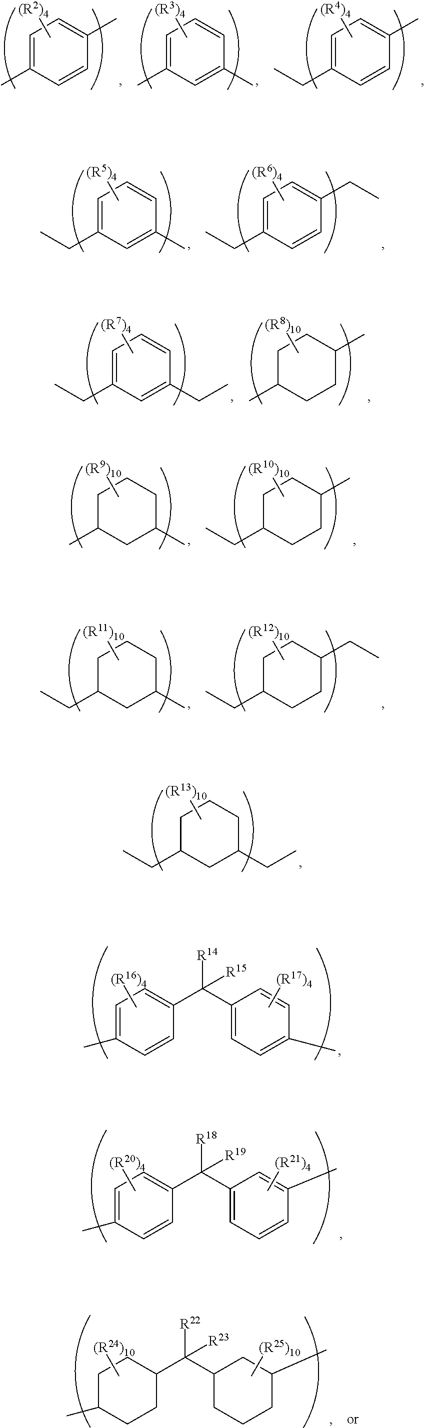

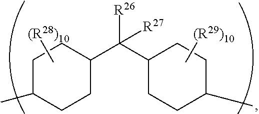

wherein R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, and R²⁹ can be independently hydrogen, C₁₋₈ alkyl group, or C₁₋₈ alkoxy group. According to embodiments of the disclosure, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸ and R²⁹ can be independently hydrogen, methyl, ethyl, propyl, butyl, pentyl, hexyl, heptyl, octyl, methoxy, ethoxy, propoxy, butoxy, pentyloxy, hexyloxy, heptyloxy, octyloxy or an isomer thereof.

According to embodiments of the disclosure, Y¹ and Y² can be independently

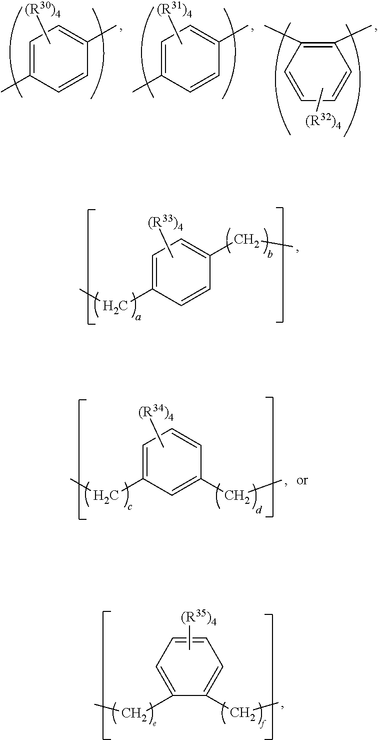

wherein R³⁰, R³¹, R³², R³³, R³⁴ and R³⁵ can be independently hydrogen, C₁₋₈ alkyl group, or C₁₋₈ alkoxy group; and, a, b, c, d, e, and f can be independently 1, 2, 3, 4, or 5. According to embodiments of the disclosure, R³⁰, R³¹, R³², R³³, R³⁴ and R³⁵ can be independently hydrogen, methyl, ethyl, propyl, butyl, pentyl, hexyl, heptyl, octyl, methoxy, ethoxy, propoxy, butoxy, pentyloxy, hexyloxy, heptyloxy, octyloxy or an isomer thereof.

According to embodiments of the disclosure, the epoxy resin monomer can be

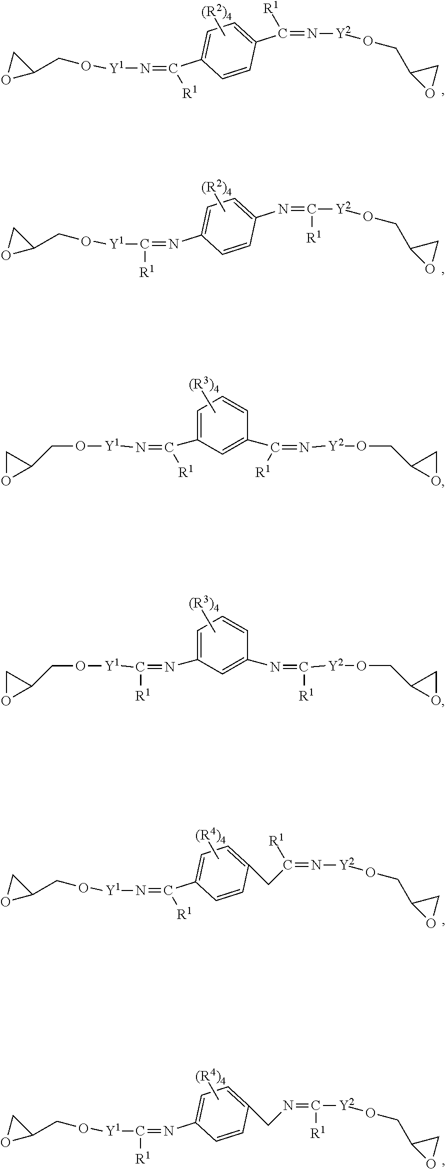

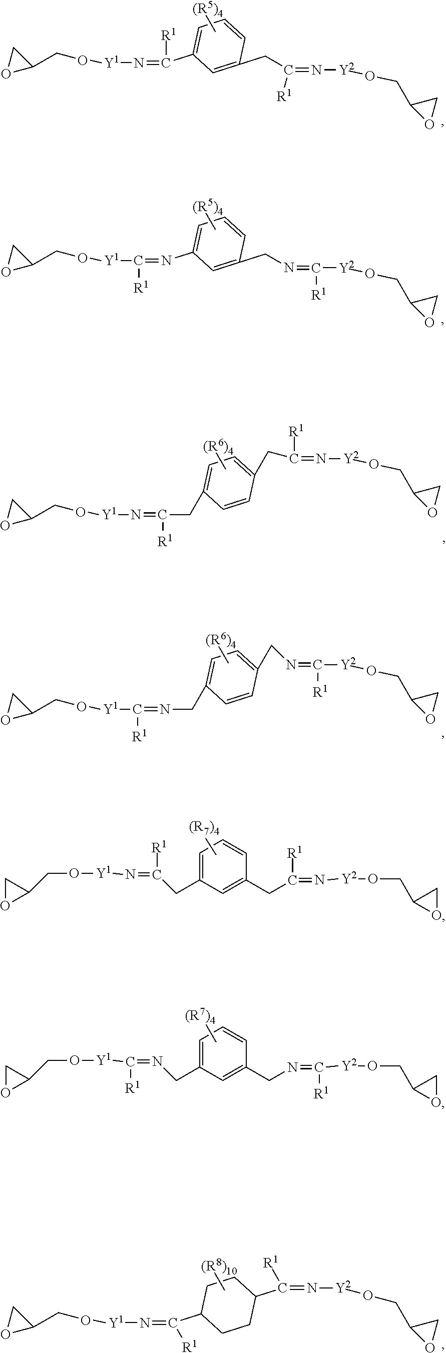

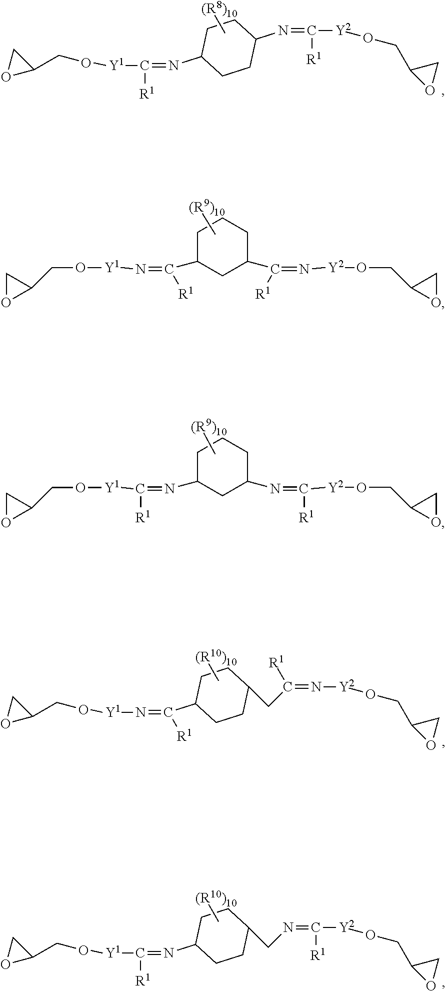

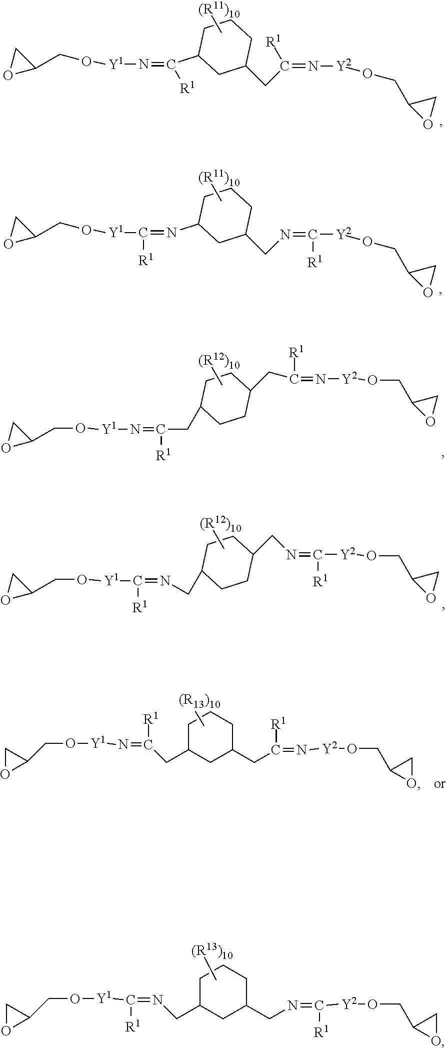

wherein R¹, R², R³, R⁴, R⁵, R⁶, R⁷, R⁸, R⁹, R¹⁰, R¹¹, R¹², R¹³ can be independently hydrogen. C₁₋₈ alkyl group, or C₁₋₈ alkoxy group; and, Y¹ and Y² are independently substituted or non-substituted C₆₋₂₄ arylene group, and Y¹ is distinct from Y².

According to embodiments of the disclosure, the epoxy resin monomer can be

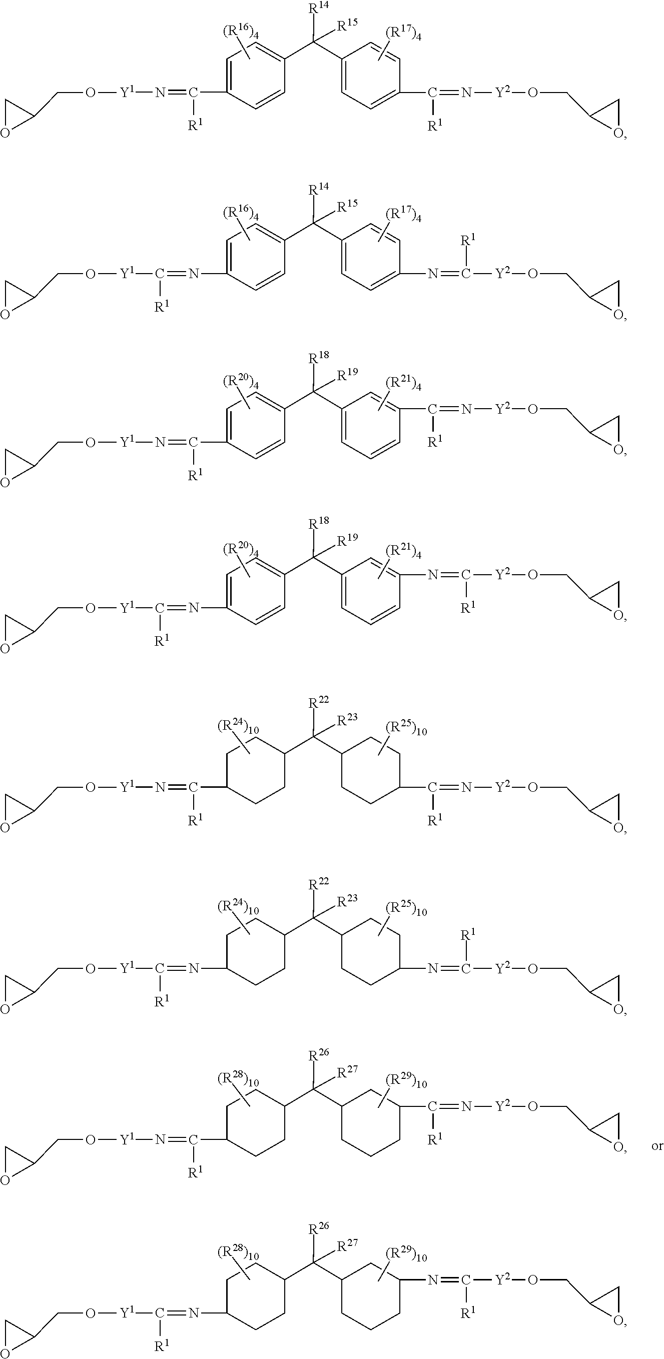

wherein R¹, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸ and R²⁹ are independently hydrogen, C₁₋₈ alkyl group, or C₁₋₈ alkoxy group; and, Y¹ and Y² are independently substituted or non-substituted C₆₋₂₄ arylene group, and Y¹ is distinct from Y².

According to embodiments of the disclosure, the epoxy resin monomer can be

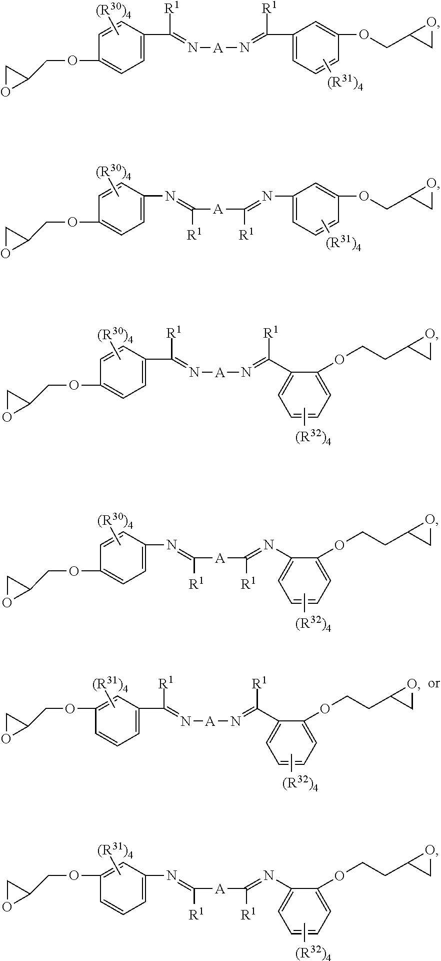

wherein A is substituted or non-substituted C₆₋₂₄ arylene group, C₃₋₁₆ cycloalkylene group, C₃₋₁₆ heteroarylene group, C₃₋₁₆ alicyclic alkylene group, or divalent C₆₋₂₅ alkylaryl group; and, R¹, R³⁰, R³¹, and R³² are independently hydrogen, C₁₋₈ alkyl group, or C₁₋₈ alkoxy group.

According to embodiments of the disclosure, the epoxy resin monomer has an epoxy equivalent weight (EEW) of about 50 g/eq to 1500 g/eq, such as 100 g/eq, 150 g/eq, 200 g/eq, 300 g/eq, 400 g/eq, 500 g/eq, 600 g/eq, 700 g/eq, 800 g/eq, 900 g/eq, 1000 g/eq, 1200 g/eq, or 1400 g/eq. According to embodiments of the disclosure, when the epoxy equivalent weight of the epoxy resin monomer is too high, the cured product of the resin composition of the disclosure exhibits poor decomposability under an acidic condition.

According to embodiments of the disclosure, the resin composition of the disclosure may further include an epoxy resin, wherein the weight ratio of the epoxy resin to the epoxy resin monomer is 1:100 to 9:1. When the amount of the epoxy resin monomer is too low, the cured product of the resin composition of the disclosure exhibits poor decomposability under an acidic condition and poor flowability.

According to embodiments of the disclosure, the epoxy resin is bisphenol A epoxy resin, bisphenol F epoxy resin, bisphenol S epoxy resin, novolac epoxy resin, naphthalene-based epoxy resin, anthracene-based epoxy resin, bisphenol A diglycidyl ether epoxy resin, ethylene glycol diglycidyl ether epoxy resin, propylene glycol diglycidyl ether epoxy resin, or 1,4-butanediol diglycidyl ether epoxy resin. According to embodiments of the disclosure, the weight average molecular weight of the epoxy resin can be about 5,000 g/mol to 2,000,000 g/mol, such as 8,000 g/mol, 10,000 g/mol, 20,000 g/mol, 50,000 g/mol, 100,000 g/mol, 300,000 g/mol, 500,000 g/mol, 1,000,000 g/mol, 1,500,000 g/mol, or 1,800,000 g/mol. The weight average molecular weight (Mw) of the epoxy resin of the disclosure can be determined by gel permeation chromatography (GPC) (based on a polystyrene calibration curve).

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

### EXAMPLE

The epoxy resin monomer having the structure represented by Formula (I) of the disclosure include the following compounds shown in Table 1.

TABLE 1

| | structure |
|---|---|
| Example 1 | 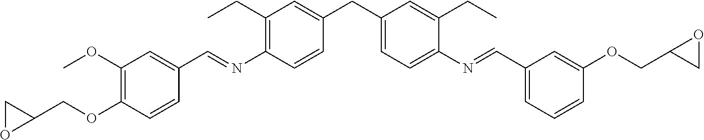 [SMILES: CCc1cc(Cc2ccc(/N=C/c3ccc(OCC4CO4)c(OC)c3)c(CC)c2)ccc1/N=C/c1cccc(OCC2CO2)c1] |
| | Epoxy resin monomer (I) |
| Example 2 | 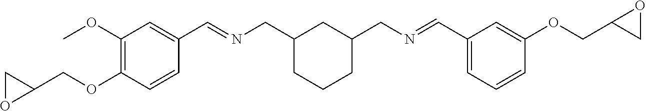 [SMILES: COc1cc(/C=N/CC2CCCC(C/N=C/c3cccc(OCC4CO4)c3)C2)ccc1OCC1CO1] |
| | Epoxy resin monomer (II) |
| Example 3 | 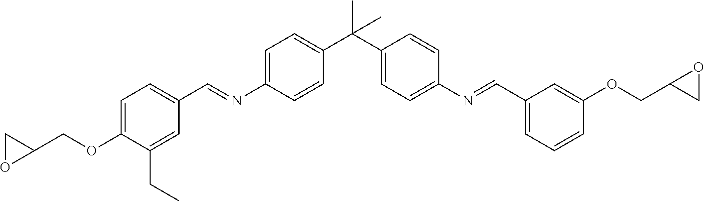 [SMILES: CCc1cc(/C=N/c2ccc(C(C)(C)c3ccc(/N=C/c4cccc(OCC5CO5)c4)cc3)cc2)ccc1OCC1CO1] |
| | Epoxy resin monomer (III) |
| Example 4 | 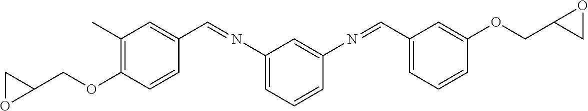 [SMILES: Cc1cc(C=Nc2cccc(N=Cc3cccc(OCC4CO4)c3)c2)ccc1OCC1CO1] |
| | epoxy resin monomer (IV) |
| Example 5 | 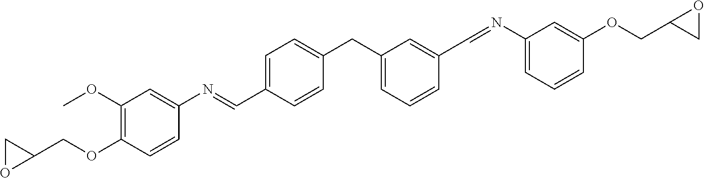 [SMILES: COc1cc(/N=C/c2ccc(Cc3cccc(C=Nc4cccc(OCC5CO5)c4)c3)cc2)ccc1OCC1CO1] |
| | Epoxy resin monomer (V) |
| Example 6 | 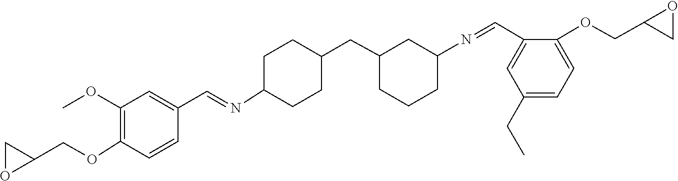 [SMILES: CCc1ccc(OCC2CO2)c(C=NC2CCCC(CC3CCC(/N=C/c4ccc(OCC5CO5)c(OC)c4)CC3)C2)c1] |
| | Epoxy resin monomer (VI) |
| Example 7 | 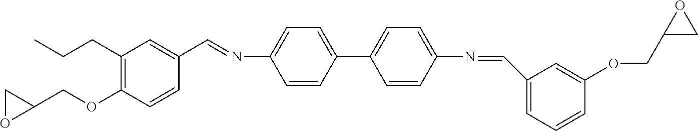 [SMILES: CCCc1cc(/C=N/c2ccc(-c3ccc(N=Cc4cccc(OCC5CO5)c4)cc3)cc2)ccc1OCC1CO1] |
| | Epoxy resin monomer (VII) |
| Example 8 | 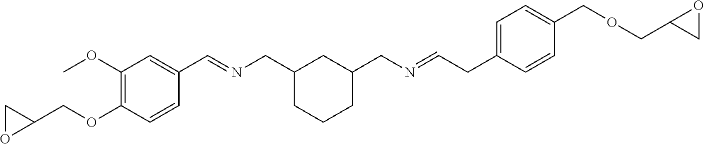 [SMILES: COc1cc(/C=N/CC2CCCC(C/N=C/Cc3ccc(COCC4CO4)cc3)C2)ccc1OCC1CO1] |
| | Epoxy resin monomer (VIII) |
| Example 9 | 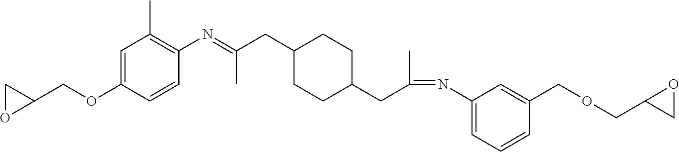 [SMILES: C/C(CC1CCC(C/C(C)=N/c2ccc(OCC3CO3)cc2C)CC1)=N/c1cccc(COCC2CO2)c1] |
| | Epoxy resin monomer (IX) |
| Example 10 | 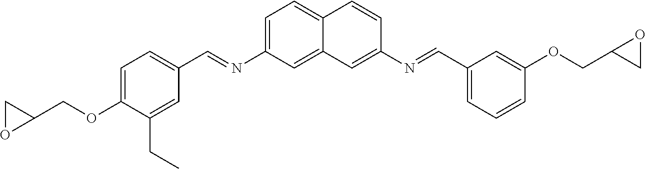 [SMILES: CCc1cc(/C=N/c2ccc3ccc(/N=C/c4cccc(OCC5CO5)c4)cc3c2)ccc1OCC1CO1] |
| | Epoxy resin monomer (X) |

According to embodiments of the disclosure, the disclosure also provides a resin film, wherein the resin film includes a cured product of the resin composition of the disclosure.

In order to clearly illustrate the method for preparing the epoxy resin monomer of the disclosure, the preparation of epoxy resin monomer disclosed in Examples 1 and 2 are described in detail below.

Preparation of Epoxy Resin Monomer of Example 1

4,4′-methylene bis(2-ethylaniline) (MOEA) (0.04 mol), vanillin (0.04 mol), 3-hydroxybenzaldehyde (MHB) (0.04 mol), p-toluenesulfonic acid (TsOH) (0.09 g), and ethanol (EtOH) (50 ml) were added into a reaction bottle, obtaining a mixture. After the mixture was allowed to react at 70° C. under nitrogen atmosphere for 5 hrs, the result was concentrated, obtaining Compound (1). The synthesis pathway of the above reaction was as follows:

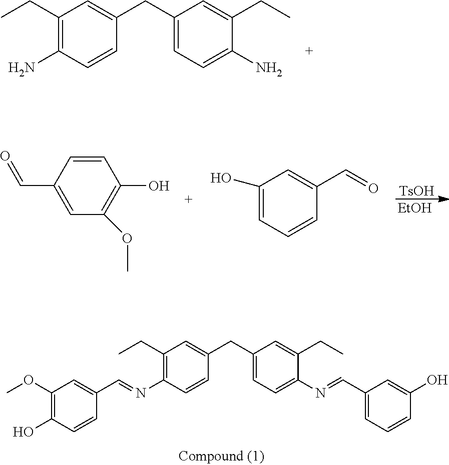

Compound (1) was characterized by nuclear magnetic resonance (¹H NMR, 400 MHz in acetone-d6) δ: 8.48 (s, 1H, —CH═N—), 8.37 (s, 1H, —CH═N—), 7.72 (s, 1H, Ar—H), 7.54 (s, 1H, Ar—H), 7.28-7.49 (m, 5H, Ar—H), 7.21-7.03 (m, 6H, Ar—H), 3.95 (s, 2H, —CH₂₋), 3.85 (s, 3H, —OCH₃), 2.75 (q, 4H, —CH₂CH₃—), 1.20 (t, 6H, —CH₂CH₃).

Next, Compound (1) (0.04 mol), epichlorohydrin (ECH) (37 g), tetrabutyl ammonium bromide (TBAB) (1.29 g), were added into a reaction bottle, obtaining a mixture. The mixture was allowed to react at 80° C. under nitrogen atmosphere for 3 hrs. After cooling to 5° C., a sodium hydroxide aqueous solution (6 g) (with a solid content of 40 wt %) was added. After stirring at 5° C. for 5 hrs, the reaction was complete. The result was concentrated, and ethyl acetate was added into the reaction bottle to dissolve the result. The obtained solution was washed three times with deionized water. The organic phase was dehydrated with magnesium sulfate, concentrated and dried, obtaining Epoxy resin monomer (I). The synthesis pathway of the above reaction was as follows:

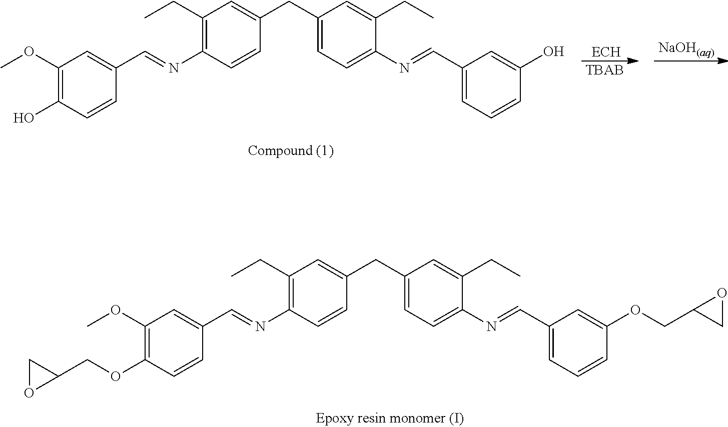

Epoxy resin monomer (I) was characterized by nuclear magnetic resonance (¹H NMR, 400 MHz in acetone-d6) δ: 8.51 (s, 1H, —CH═N—), 8.40 (s, 1H, —CH═N—), 7.75 (s, 1H, Ar—H), 7.68 (s, 1H, Ar—H), 7.52 (d, 1H, Ar—H), 7.47 (m, 1H, Ar—H), 7.25-6.1 (9H, Ar—H), 4.42-2.75 (10H, —CH₂—; —CH— and —CH₂— of oxirane), 3.98 (s, 2H, —CH₂₋), 3.95 (s, 6H, —OCH₃), 2.75 (m, 4H, —CH₂CH₃—), 1.20 (t, 6H, —CH₂CH₃).

Preparation of Epoxy Resin Monomer of Example 2

1,3-bis(aminomethyl) cyclohexane (1,3-BAC) (0.04 mol), vanillin (0.04 mol), 3-hydroxybenzaldehyde (MHB) (0.04 mol), p-toluenesulfonic acid (TsOH) (0.09 g), and ethanol (EtOH) (50 ml) were added into a reaction bottle, obtaining a mixture. The mixture was allowed to react at 70° C. under nitrogen atmosphere for 5 hrs. After cooling the reaction bottle to room temperature, the result was concentrated, obtaining Compound (2). The synthesis pathway of the above reaction was as follows:

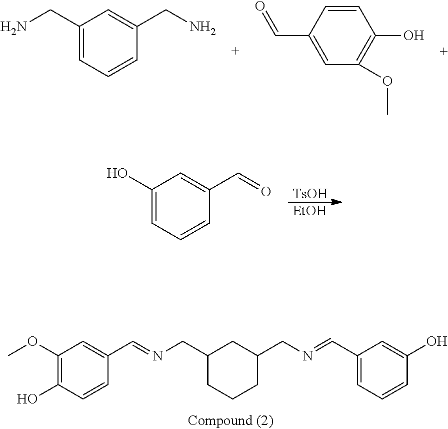 [SMILES: "EtOH" ; NCc1cccc(CN)c1 ; COc1cc(C=O)ccc1O ; O=Cc1cccc(O)c1 ; "TsOH" ; COc1cc(/C=N/CC2CCCC(C/N=C/c3cccc(O)c3)C2)ccc1O]

Compound (2) was characterized by nuclear magnetic resonance (¹H NMR, 400 MHz in acetone-d6) δ: 8.21 (S, 1H, —CH═N—), 8.15 (S, 1H, —CH═N—), 7.48 (s, 1H, Ar—H), 7.30 (s, 1H, Ar—H), 7.25-7.09 (m, 3H, Ar—H), 6.89-6.79 (m, 2H, Ar—H), 3.81 (s, 3H, —OCH₃), 3.54-3.36 (m, 2H, —CH₂—), 1.99-1.22 (m, 6H, —CH₂—), 1.0-0.72 (m, 2H, —CH₂—).

Next, Compound (2) (0.04 mol), epichlorohydrin (ECH) (37 g), tetrabutyl ammonium bromide (TBAB) (1.29 g), were added into a reaction bottle, obtaining a mixture. After heating the reaction bottle to 80° C. under nitrogen atmosphere, the mixture was stirred for 3 hrs. After cooling the reaction bottle to a temperature less than 5° C., sodium hydroxide aqueous solution (6 g) (with a solid content of 40 wt %) to the reaction bottle. After stirring at 5° C. for 5 hrs, the mixture was concentrated, and ethyl acetate was added into the reaction bottle to dissolve the result. The obtained solution was washed three times by deionized water. The organic phase was dehydrated with magnesium sulfate, concentrated and dried, obtaining Epoxy resin monomer (II). The synthesis pathway of the above reaction was as follows:

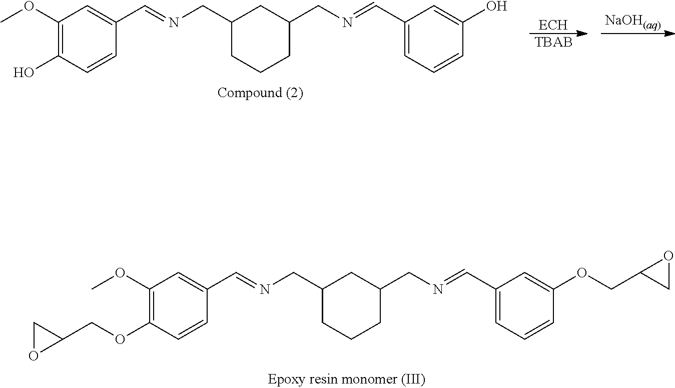

Epoxy resin monomer (II) was characterized by nuclear magnetic resonance (¹H NMR, 400 MHz in acetone-d6) δ: ¹H NMR (acetone-d6; 400 MHz) δ: 8.19 (m, 1H, —CH═N—), 8.14 (m, 1H, —CH═N—), 7.45 (s, 1H, Ar—H), 7.30 (s, 1H, Ar—H), 7.26-7.10 (m, 3H, Ar—H), 6.93-6.84 (m, 2H, Ar—H), 4.42-2.75 (m, 10H, —O—CH₂—; —CH— and —CH₂— of oxirane), 3.81 (s, 3H, —OCH₃), 3.54-3.36 (m, 4H, —CH₂—), 1.99-1.22 (m, 6H, —CH₂—), 1.0-0.72 (m, 2H, —CH₂—).

### Comparative Example 1

4,4′-methylene bis(2-ethylaniline) (MOEA) (0.04 mol), vanillin (0.08 mol), p-toluenesulfonic acid (TsOH) (0.09 g), and ethanol (EtOH) (50 ml) were added into a reaction bottle, obtaining a mixture. The mixture was allow to react at 70° C. under nitrogen atmosphere for 5 hrs. After cooling the reaction bottle to room temperature, the result was concentrated, obtaining Compound (3). The synthesis pathway of the above reaction was as follows:

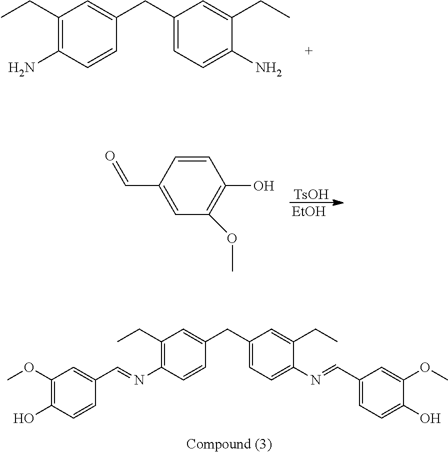

Compound (3) was characterized by nuclear magnetic resonance (¹H NMR, 400 MHz in acetone-d6) δ: 8.47 (s, 2H, —CH═N—), 7.65 (s, 2H, Ar—H), 7.43 (dd, 2H, Ar—H), 7.16 (s, 2H, Ar—H), 7.10 (dd, 2H, Ar—H), 7.04 (dd, 2H, Ar—H), 6.75 (dd, 2H, Ar—H), 4.42 (dd, 2H, —CH₂—), 3.90 (s, 6H, —OCH₃), 2.75 (m, 4H, —CH₂CH₃), 1.15 (t, 6H, —CH₂CH₃).

Next, Compound (3) (0.04 mol), epichlorohydrin (ECH) (37 g), and tetrabutyl ammonium bromide (TBAB) (1.29 g) were added into a reaction bottle, obtaining a mixture. The mixture was allowed to react at 80° C. under nitrogen atmosphere for 3 hrs. After cooling the reaction bottle reaction bottle to a temperature less than 5° C., sodium hydroxide aqueous solution (6 g) (with a solid content of 40 wt %) was added into the reaction bottle. After stirring at 5° C. for 5 hrs, the mixture was concentrated, and ethyl acetate was added into the reaction bottle to dissolve the result. The obtained solution was washed three times by deionized water. The organic phase was dehydrated with magnesium sulfate, concentrated and dried, obtaining Epoxy resin monomer (XI). The synthesis pathway of the above reaction was as follows:

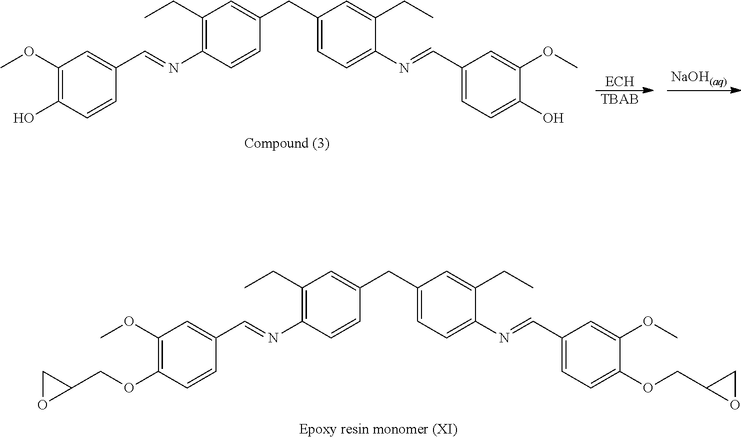

Epoxy resin monomer (XI) was characterized by nuclear magnetic resonance (¹H NMR, 400 MHz in acetone-d6) δ: 8.47 (s, 2H, —CH═N—), 7.70 (s, 2H, Ar—H), 7.42 (dd, 2H, Ar—H), 7.15 (s, 2H, Ar—H), 7.10 (dd, 2H, Ar—H), 7.05 (dd, 2H, Ar—H), 6.85 (dd, 2H, Ar—H), 4.42 (dd, 2H, —CH₂—), 3.90 (s, 6H, —OCH₃), 3.88 (dd, 2H, —O—CH₂—), 3.35 (dt, 2H, —CH— of oxirane), 2.85-2.78 (m, 4H, —CH₂— of oxirane), 2.75 (m, 4H, —CH₂CH₃), 1.15 (t, 6H, —CH₂CH₃).

The melting point and the epoxy equivalent weight of Epoxy resin monomer (I) and (II) of Examples 1 and 2 and Epoxy resin monomer (XI) of Comparative Example 1 were measured and the results are shown in Table 2. The melting point of epoxy resin monomer is measured by differential scanning calorimetry (DSC); and, the epoxy equivalent weight of epoxy resin monomer is measured by a method according to ASTM D1652.

TABLE 2

| | melting point | epoxy equivalent |
|---|---|---|
| | (° C.) | weight (g/eq) |
| Epoxy resin monomer (I) | <25 | 378 |
| Epoxy resin monomer (II) | <25 | 285 |
| Epoxy resin monomer (XI) | 67 | 326 |

As shown in Table 2, since Epoxy resin monomer (XI) is a symmetrical epoxy resin monomer (i.e. the monomer has a symmetrical chemical structure), Epoxy resin monomer (XI) exhibits high crystallinity, resulting in that Epoxy resin monomer (XI) has a higher melting point (i.e. Epoxy resin monomer (XI) is solid at room temperature). In addition, Epoxy resin monomers (I) and (II) of the disclosure are asymmetrical epoxy resin monomer (i.e. the monomers have an asymmetrical chemical structure), resulting in that Epoxy resin monomers (I) and (II) have a lower melting point and are liquid at room temperature.

Preparation of Resin Composition

### Example 11

Epoxy resin monomer (I) was mixed with 2-methyl imidazole (2MI) (serving as a hardener), obtaining Resin composition (1). Herein, the equivalent ratio of Epoxy resin monomer (I) to 2-methyl imidazole was 1:1.

### Example 12

Epoxy resin monomer (I) was mixed with poly(propylene glycol) bis(2-aminopropyl ether) (with a molecular weight of 230) (with a trade number of Jeffamine® D-230) (serving as a hardener), obtaining Resin composition (2). Herein, the equivalent ratio of Epoxy resin monomer (I) to poly(propylene glycol) bis(2-aminopropyl ether) was 1:1.

### Example 13

Epoxy resin monomer (I), methyltetrahydrophthalic anhydride (MTHPA) (serving as a hardener), 1,8-diazabicyclo[5.4.0]undec-7-ene (DBU), and 2-ethylhexanoic acid were mixed, obtaining Resin composition (3). Herein, the equivalent ratio of Epoxy resin monomer (I) to methyltetrahydrophthalic anhydride (MTHPA) was 1:1, the amount of 1,8-diazabicyclo[5.4.0]undec-7-ene (DBU) was 0.25 phr, and the amount of 2-ethylhexanoic acid was 0.25 phr (based on the weight of Epoxy resin monomer (I)).

### Example 14

Epoxy resin monomer (I), bisphenol F epoxy resin (with a trade number of EPICLON® EXA-830LVP), methyltetrahydrophthalic anhydride (MTHPA) (serving as a hardener), 1,8-diazabicyclo[5.4.0]undec-7-ene (DBU), and 2-ethylhexanoic acid were mixed, obtaining Resin composition (4). Herein, the equivalent ratio of Epoxy resin monomer (I), bisphenol F epoxy resin, and methyltetrahydrophthalic anhydride (MTHPA) was 1:1:2, the amount of 1,8-diazabicyclo[5.4.0]undec-7-ene (DBU) was 0.25 phr, and the amount of 2-ethylhexanoic acid was 0.25 phr (based on the weight of Epoxy resin monomer (I)).

### Example 15

Example 15 was performed in the same manner as in Example 14, except that the equivalent ratio of Epoxy resin monomer (I), bisphenol F epoxy resin, and methyltetrahydrophthalic anhydride (MTHPA) was adjusted from 1:1:2 to 0.5:1.5:2, obtaining Resin composition (5).

### Example 16

Example 16 was performed in the same manner as in Example 11, except that the equivalent ratio of Epoxy resin monomer (I) to 2-methyl imidazole was adjusted from 1:1 to 10:7, obtaining Resin composition (6).

### Example 17

Example 17 was performed in the same manner as in Example 12, except that the equivalent ratio of Epoxy resin monomer (I) to poly(propylene glycol) bis(2-aminopropyl ether) was adjusted from 1:1 to 10:7, obtaining Resin composition (7).

### Example 18

Example 18 was performed in the same manner as in Example 13, except that the equivalent ratio of Epoxy resin monomer (I) to methyltetrahydrophthalic anhydride (MTHPA) was adjusted from 1:1 to 10:7, obtaining Resin composition (8).

### Example 19

Example 19 was performed in the same manner as in Example 11, except that Epoxy resin monomer (I) was replaced with Epoxy resin monomer (II), obtaining Resin composition (9).

### Example 20

Example 20 was performed in the same manner as in Example 12, except that Epoxy resin monomer (I) was replaced with Epoxy resin monomer (II), obtaining Resin composition (10).

### Example 21

Example 21 was performed in the same manner as in Example 13, except that Epoxy resin monomer (I) was replaced with Epoxy resin monomer (II), obtaining Resin composition (11).

### Example 22

Example 22 was performed in the same manner as in Example 14, except that Epoxy resin monomer (I) was replaced with Epoxy resin monomer (II), obtaining Resin composition (12).

### Example 23

Example 23 was performed in the same manner as in Example 15, except that Epoxy resin monomer (I) was replaced with Epoxy resin monomer (II), obtaining Resin composition (13).

### Comparative Example 2

Comparative Example 2 was performed in the same manner as in Example 14, except that Epoxy resin monomer (I) was replaced with Epoxy resin monomer (XI), obtaining Resin composition (14).

### Comparative Example 3

Comparative Example 3 was performed in the same manner as in Example 15, except that Epoxy resin monomer (I) was replaced with Epoxy resin monomer (XI), obtaining Resin composition (15).

### Comparative Example 4

Comparative Example 4 was performed in the same manner as in Example 11, except that Epoxy resin monomer (I) was replaced with bisphenol F epoxy resin (with a trade number of EPICLON® EXA-830LVP), obtaining Resin composition (16).

### Comparative Example 5

Comparative Example 5 was performed in the same manner as in Example 12, except that Epoxy resin monomer (I) was replaced with bisphenol F epoxy resin (with a trade number of EPICLON® EXA-830LVP), obtaining Resin composition (17).

### Comparative Example 6

Comparative Example 6 was performed in the same manner as in Example 13, except that Epoxy resin monomer (I) was replaced with bisphenol F epoxy resin (with a trade number of EPICLON® EXA-830LVP), obtaining Resin composition (18).

Flowability Test

The flowability of Resin compositions (2)-(5) of Example 12-15 and Resin compositions (14)-(15) of Comparative Example 2 and 3 was evaluated, and the results are shown in Table 3. Herein, the flowability was evaluated by the viscosity measured at a temperature 25° C. and a rotating rate of 10 rpm. When the viscosity is less than 55,000 cps, it means the resin composition exhibits great flowability and marked with O. When the viscosity is between 55,000 cps and 65,000 cps, it means that the resin composition exhibits acceptable flowability and marked with Δ. When the viscosity is greater than 65,000 cps, it means that the resin composition exhibits poor flowability or non-flowability and marked with X.

TABLE 3

| | epoxy resin | epoxy | | |
|---|---|---|---|---|
| | monomer | resin | hardener | flowability |
| Resin | Epoxy resin | — | polypropylene | ◯ |
| composition | monomer | | glycol) bis(2- | |
| (2) | (I) | | aminopropyl | |
| | (1 eq) | | ether) | |
| | | | (1 eq) | |
| Resin | Epoxy resin | — | MTHPA | ◯ |
| composition | monomer | | (1 eq) | |
| (3) | (I) | | | |
| | (1 eq) | | | |
| Resin | Epoxy resin | bisphenol | MTHPA | ◯ |
| composition | monomer | F epoxy | (1 eq) | |
| (4) | (I) | resin | | |
| | (0.5 eq) | (0.5 eq) | | |
| resin | Epoxy resin | bisphenol | MTHPA | X |
| composition | monomer | F epoxy | (1 eq) | |
| (14) | (XI) | resin | | |
| | (0.5 eq) | (0.5 eq) | | |
| Resin | Epoxy resin | bisphenol | MTHPA | ◯ |
| composition | monomer | F epoxy | (1 eq) | |
| (5) | (I) | resin | | |
| | (0.25 eq) | (0.75 eq) | | |
| Resin | Epoxy resin | bisphenol | MTHPA | Δ |
| composition | monomer | F epoxy | (1 eq) | |
| (15) | (XI) | resin | | |
| | (0.25 eq) | (0.75 eq) | | |

Since the epoxy resin monomer of the disclosure has an asymmetrical chemical structure, the epoxy resin monomer of the disclosure has a lower melting point such that the epoxy resin monomer is liquid at room temperature. Therefore, the resin composition employing the epoxy resin monomer of the disclosure exhibits flowability and is suitable for serving as packaging material. In comparison with Resin composition (4), Resin compositions (14) and (15) employ Epoxy resin monomer (XI) (symmetrical epoxy resin monomer) substituting for with Epoxy resin monomer (I) (asymmetrical epoxy resin monomer), and the flowability of Resin compositions (14) and (15) is obviously reduced (as shown in Table 3), resulting in reducing processability of the resin composition.

Properties Analysis of Cured Product of Resin Composition

The resin composition (1) and the cured product thereof were analyzed by Fourier-transform infrared spectroscopy (FT-IR). Epoxy resin monomer (I) had a peak at 911 cm⁻¹ (representing the oxiranyl group thereof) and the peak at 911 cm¹ of Epoxy resin monomer (I) was vanished after ring-opening crosslinking reaction.

The cured products of Resin compositions (1)-(3) of Examples 11-13 and Resin compositions (16)-(18) of Comparative Examples 4-6 were subjected to the thermal tolerance evaluation and the acid-decomposition test, and the results are shown in Table 4. The thermal tolerance evaluation included measuring the thermal decomposition temperature (Td) (the temperature at which that 5% weight loss was observed) of the cured product (5 mg) of resin composition by thermogravimetric analysis (TGA) under nitrogen atmosphere. The acid-decomposition test included following steps. First, sulfuric acid, water and tetrahydrofuran (THF) were mixed, obtaining a sulfuric acid solution with a concentration of 0.2M (the volume ratio of water to tetrahydrofuran (THF) was 2:8). Next, the cured product (25 mg) of the resin composition was disposed in the sulfuric acid solution 5 ml, and the result was stirred at 65° C. Finally, after a period of time, the results were observed to determine whether the cured product was decomposed or dissolved in the sulfuric acid solution. When the cured product was completely dissolved in the sulfuric acid solution, it was marked with ⊚. When the cured product was completely broken into pieces and partially dissolved in the sulfuric acid solution, it was marked with ◯. When the cured product could not be decomposed or dissolved in the sulfuric acid solution, it was marked with X. After analyzing the cured product of Resin composition (1) and the result thereof via the acid-decomposition test by Fourier-transform infrared spectroscopy (FT-IR), the result shows that the signal intensity of the C═N character peak at 1625 cm¹ of the cured product of Resin composition (1) is reduced and a C═O character peak at 1647 cm¹ is observed after the acid-decomposition test.

It means that the imino group of the cured product of the resin composition of the disclosure is broken under an acidic condition, thereby facilitating the decomposition of the cured product of Resin composition (1).

TABLE 4

| | | Resin | Resin | Resin | Resin | Resin | Resin |
|---|---|---|---|---|---|---|---|
| | | composition | composition | composition | composition | composition | composition |
| | | (1) | (16) | (2) | (17) | (3) | (18) |
| epoxy resin | | Epoxy resin | — | Epoxy resin | — | Epoxy resin | — |
| monomer | | monomer (I) | | monomer (I) | | monomer (I) | |
| | | (1 eq) | | (1 eq) | | (1 eq) | |
| epoxy resin | | — | bisphenol | — | bisphenol | — | bisphenol |
| | | | F epoxy | | F epoxy | | F epoxy |
| | | | resin (1 eq) | | resin (1 eq) | | resin (1 eq) |
| hardener | | 2-methyl | 2-methyl | poly(propylene | poly(propylene | MTHPA | MTHPA |
| | | imidazole | imidazole | glycol) | glycol) | (1 eq) | (1 eq) |
| | | (1 eq) | (1 eq) | bis(2- | bis(2- | | |
| | | | | aminopropyl | aminopropyl | | |
| | | | | ether) | ether) | | |
| | | | | (1 eq) | (1 eq) | | |
| thermal decomposition | | 302 | 287 | 289 | 316 | 310 | 338 |
| temperature (Td) | | | | | | | |
| (5% weight loss) | | | | | | | |
| (° C.) | | | | | | | |
| acid- | 0.5 hr | ⊚ | X | X | X | ◯ | X |
| decomposition | 2 hrs | ⊚ | X | ◯ | X | ⊚ | X |
| test | 4 hrs | ⊚ | X | ◯ | X | ⊚ | X |
| | 18 hrs | ⊚ | X | ◯ | X | ⊚ | X |
| | 24 hrs | ⊚ | X | ⊚ | X | ⊚ | X |

As shown in Table 4, since the thermal decomposition temperature (Td) of the cured products of Resin compositions (1)-(3) of the disclosure is greater than 280° C., it means that the cured product of the resin composition of the disclosure exhibits great thermal tolerance. In comparison with resin composition (16)-(18), since Resin composition (1)-(3) of the disclosure employs Epoxy resin monomer (I) (asymmetrical epoxy resin monomer) substituting for conventional bisphenol F epoxy resin (symmetrical epoxy resin), the cured product of Resin composition (1) (3) can be decomposed at 65° C. under an acidic condition.

The cured products of Resin compositions (4), (5), (12) and (13) of Examples 14, 15, 22 and 23 were subjected to the thermal tolerance evaluation and the acid-decomposition test, and the results are shown in Table 5.

TABLE 5

| | | Resin | Resin | Resin | Resin |
|---|---|---|---|---|---|
| | | composition | composition | composition | composition |
| | | (4) | (12) | (5) | (13) |
| epoxy resin | | Epoxy resin | Epoxy resin | Epoxy resin | Epoxy resin |
| monomer | | monomer (I) | monomer (II) | monomer (I) | monomer (II) |
| | | (0.5 eq) | (0.5 eq) | (0.25 eq) | (0.25 eq) |
| epoxy resin | | bisphenol F | bisphenol F | bisphenol F | bisphenol F |
| | | epoxy resin | epoxy resin | epoxy resin | epoxy resin |
| | | (0.5 eq) | (0.5 eq) | (0.75 eq) | (0.75 eq) |
| hardener | | MTHPA | MTHPA | MTHPA | MTHPA |
| | | (1 eq) | (1 eq) | (1 eq) | (1 eq) |
| thermal decomposition temperature | | 312 | 309 | 320 | 315 |
| (Td) (5% weight loss) (° C.) | | | | | |
| acid- | 0.5 hr | X | X | X | X |
| decomposition | 2 hrs | ◯ | ◯ | X | X |
| test | 4 hrs | ⊚ | ⊚ | X | X |
| | 18 hrs | ⊚ | ⊚ | ◯ | ◯ |
| | 24 hrs | ⊚ | ⊚ | ⊚ | ⊚ |

As shown in Table 5, since the thermal decomposition temperature (Td) of the cured products of Resin compositions (12) and (13) is greater than 300° C., it means that the cured product of the resin composition of the disclosure exhibits great thermal tolerance. In addition, the cured products of Resin compositions (4), (5), (12) and (13) can be decomposed at 65° C. under an acidic condition.

The cured products of Resin compositions (6)-(11) of Examples 16-21 were subjected to the thermal tolerance evaluation and the acid-decomposition test, and the results are shown in Table 6.

TABLE 6

| | | Resin | Resin | Resin | Resin | Resin | Resin |
|---|---|---|---|---|---|---|---|
| | | composition | composition | composition | composition | composition | composition |
| | | (6) | (7) | (8) | (9) | (10) | (11) |
| epoxy resin | | Epoxy resin | Epoxy resin | Epoxy resin | Epoxy resin | Epoxy resin | Epoxy resin |
| monomer | | monomer (I) | monomer (I) | monomer (I) | monomer (I) | monomer (II) | monomer (II) |
| | | (1 eq) | (1 eq) | (1 eq) | (1 eq) | (1 eq) | (1 eq) |
| hardener | | 2-methyl | poly(propylene | MTHPA | 2-methyl | poly(propylene | MTHPA |
| | | imidazole | glycol) | (0.7 eq) | imidazole | glycol) | (1 eq) |
| | | (0.7 eq) | bis(2- | | (1 eq) | bis(2- | |
| | | | aminopropyl | | | aminopropyl | |
| | | | ether) | | | ether) | |
| | | | (0.7 eq) | | | (1 eq) | |
| thermal decomposition | | 308 | 295 | 305 | 300 | 305 | 310 |
| temperature (Td) | | | | | | | |
| (5% weight loss) | | | | | | | |
| (° C.) | | | | | | | |
| acid- | 0.5 hr | ⊚ | X | ◯ | ⊚ | X | ◯ |
| decomposition | 2 hrs | ⊚ | ◯ | ⊚ | ⊚ | ◯ | ⊚ |
| test | 4 hrs | ⊚ | ◯ | ⊚ | ⊚ | ◯ | ⊚ |
| | 18 hrs | ⊚ | ⊚ | ⊚ | ⊚ | ◯ | ⊚ |
| | 24 hrs | ⊚ | ⊚ | ⊚ | ⊚ | ⊚ | ⊚ |

As shown in Table 6, since the thermal decomposition temperature (Td) of the cured products of Resin compositions (6)-(11) is greater than 290° C., it means that the cured product of the resin composition of the disclosure exhibits great thermal tolerance. In addition, the cured products of Resin compositions (6)-(11) can be decomposed at 65° C. under an acidic condition.

Accordingly, the cured product of the resin composition of the disclosure can be decomposed at a relatively low temperature (equal to or less than 80° C.) under an acidic condition, thereby solving the problems of thermosetting resin that does not decompose easily. In addition, since the epoxy resin monomer of the disclosure has a lower melting point (less than room temperature), the resin composition of the disclosure employing the same exhibits greatly improved flowability.

It will be clear that various modifications and variations can be made to the disclosed methods and materials. It is intended that the specification and examples be considered as exemplary only, with the true scope of the disclosure being indicated by the following claims and their equivalents.

What is claimed is:

1. A resin composition, comprising:
a hardener; and
an epoxy resin monomer, wherein the epoxy resin monomer has a structure represented by Formula (I) 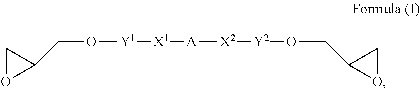
wherein A is substituted or non-substituted C₆₋₂₄ arylene group, substituted or non-substituted C₃₋₁₆ cycloalkylene group, substituted or non-substituted C₃₋₁₆ heteroarylene group, substituted or non-substituted C₃₋₁₆ alicyclic alkylene group, or substituted or non-substituted divalent C₆₋₂₅ alkylaryl group; X¹ and X² are independently 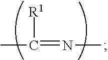
Y¹ and Y² are independently substituted or non-substituted C₆₋₂₄ arylene group, and Y¹ is different from Y²; and, R¹ is hydrogen, C₁₋₈ alkyl group, or C₁₋₈ alkoxy group, wherein the weight ratio of the curing agent to the epoxy resin monomer is 1:100 to 1:1.

2. The resin composition as claimed in claim 1, wherein X¹ is bonded to Y¹ via a carbon atom on one side and bonded to A via a nitrogen atom on the other side, and X² is bonded to Y² via a carbon atom on one side and bonded to A via a nitrogen atom on the other side.

3. The resin composition as claimed in claim 1, wherein X¹ is bonded to Y¹ via a nitrogen atom on one side and bonded to A via a carbon atom on the other side, and X² is bonded to Y² via a nitrogen atom on one side and bonded to A via a carbon atom on the other side.

4. The resin composition as claimed in claim 1, wherein A is 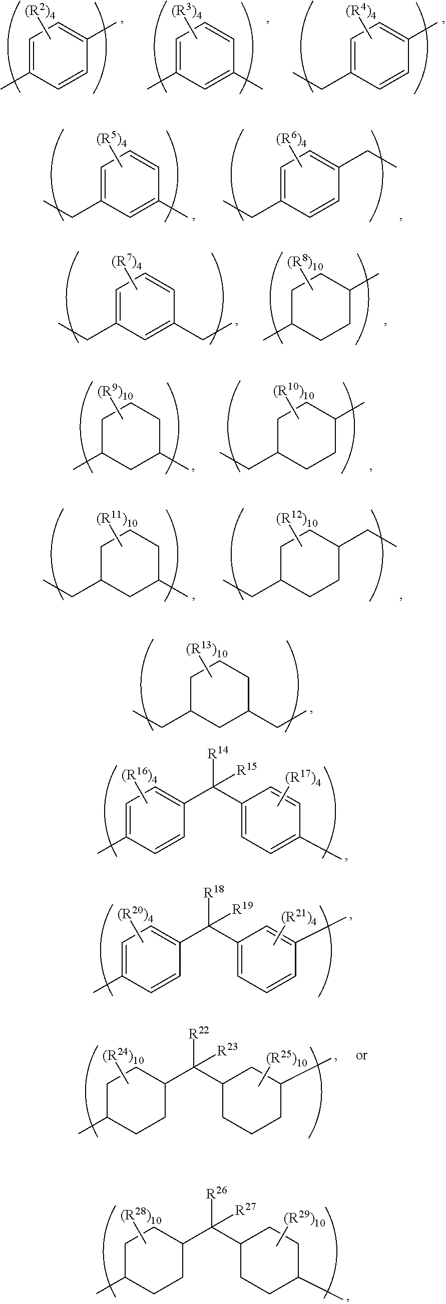
wherein R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, and R²⁹ are independently hydrogen, C₁₋₈ alkyl group, or C₁₋₈ alkoxy group.

5. The resin composition as claimed in claim 1, wherein Y¹ and Y² are independently 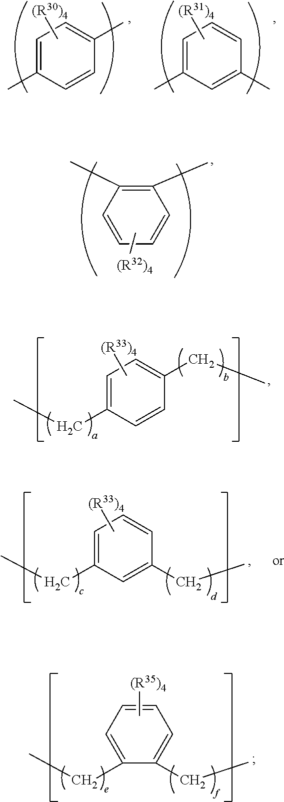
R³⁰, R³¹, R³², R³³, R³⁴, and R³⁵ are independently hydrogen, C₁₋₈ alkyl group, or C₁₋₈ alkoxy group; and a, b, c, d, e, and f are independently 1, 2, 3, 4, or 5.

6. The resin composition as claimed in claim 1, wherein the epoxy resin monomer is 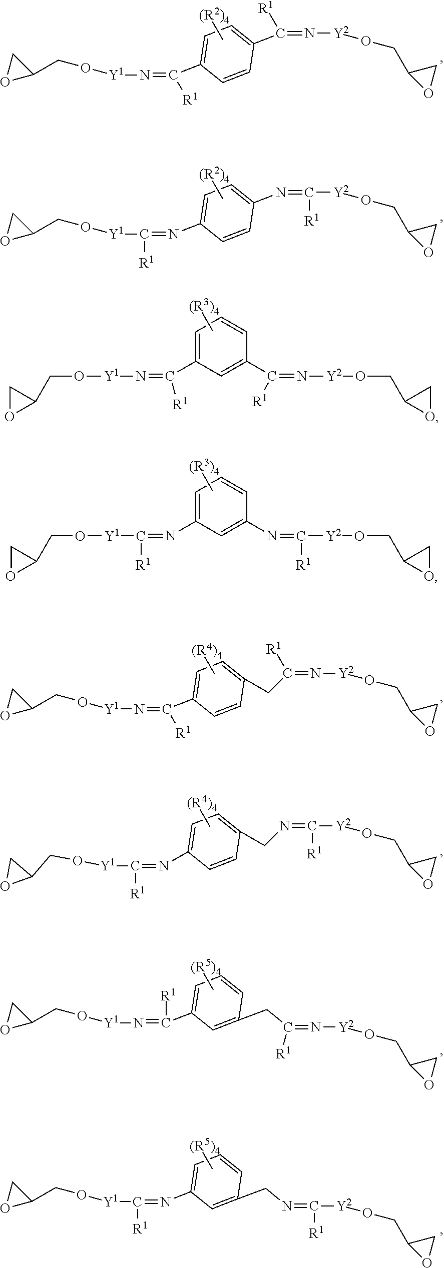 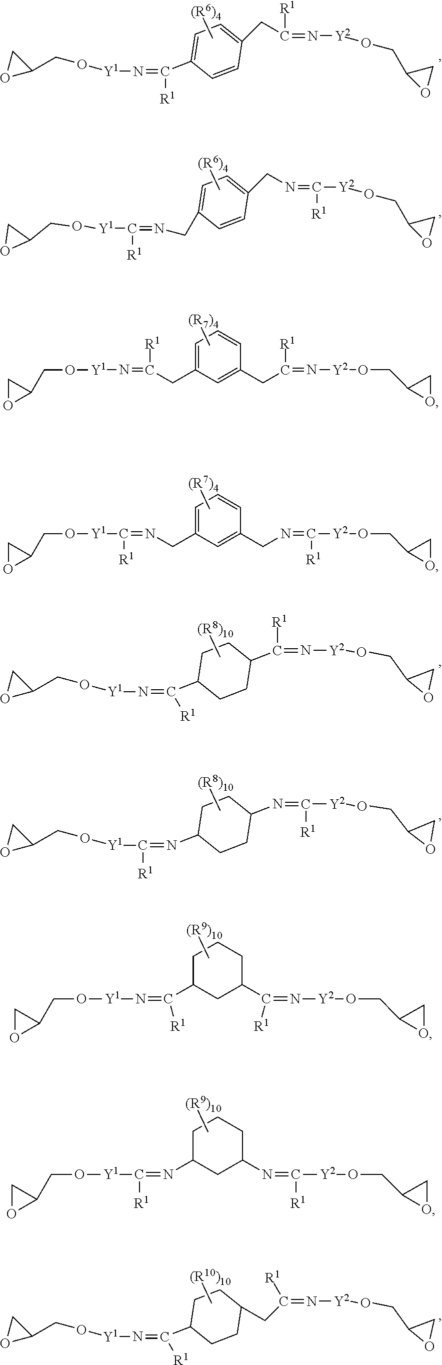 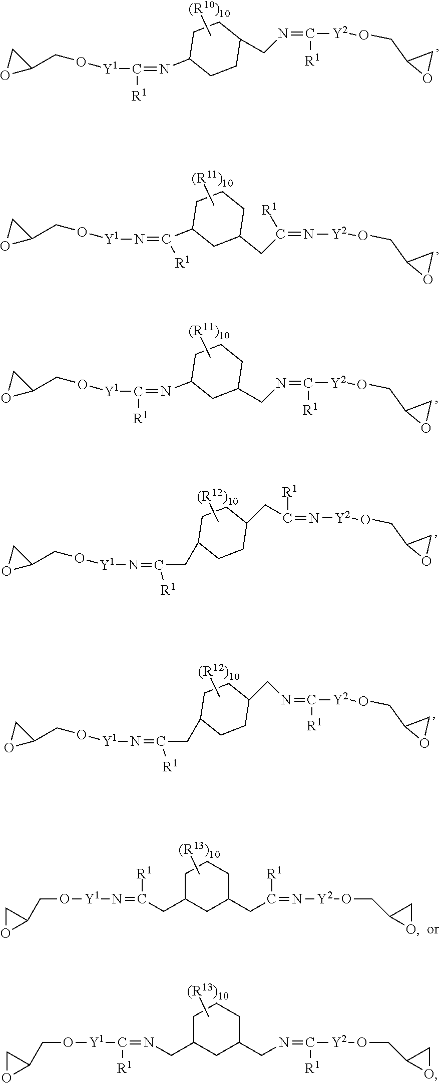
wherein R¹, R², R³, R⁴, R⁵, R⁶, R⁷, R⁸, R⁹, R¹⁰, R¹¹, R¹², and R¹³ are independently hydrogen, C₁₋₈ alkyl group, or C₁₋₈ alkoxy group; Y¹ and Y² are independently substituted or non-substituted C₆₋₂₄ arylene group; and Y¹ is different from Y².

7. The resin composition as claimed in claim 1, wherein the epoxy resin monomer is 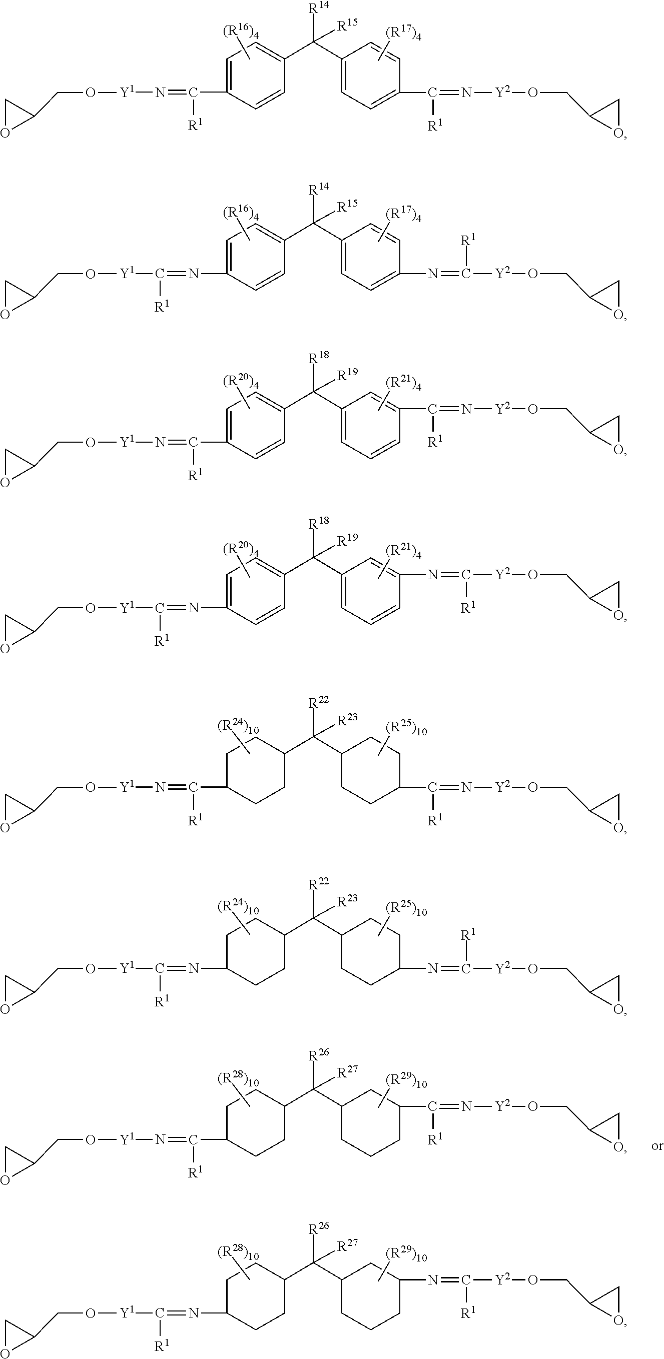
wherein R¹, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, and R²⁹ are independently hydrogen, C₁₋₈ alkyl group, or C₁₋₈ alkoxy group; Y¹ and Y² are independently substituted or non-substituted C₆₋₂₄ arylene group; and Y¹ is distinct from Y².

8. The resin composition as claimed in claim 1, wherein the epoxy resin monomer is 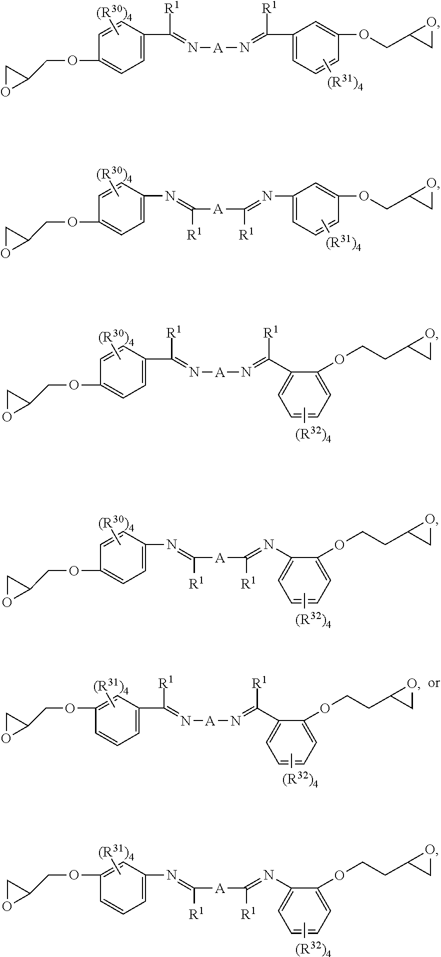
wherein A is substituted or non-substituted C₆₋₂₄ arylene group, substituted or non-substituted C₃₋₁₆ cycloalkylene group, substituted or non-substituted C₃₋₁₆ heteroarylene group, substituted or non-substituted C₃₋₁₆ alicyclic alkylene group, or substituted or non-substituted divalent C₆₋₂₅ alkylaryl group; and, R¹, R³⁰, R³¹ and R³² are independently hydrogen, C₁₋₈ alkyl group, or C₁₋₈ alkoxy group.

9. The resin composition as claimed in claim 1, wherein the epoxy resin monomer has an epoxy equivalent weight (EEW) of 50 g/eq to 1500 g/eq.

10. The resin composition as claimed in claim 1, further comprising:
an epoxy resin, wherein a weight ratio of the epoxy resin to the epoxy resin monomer is 1:100 to 9:1.

11. The resin composition as claimed in claim 10, wherein the epoxy resin is bisphenol A epoxy resin, bisphenol F epoxy resin, bisphenol S epoxy resin, novolac epoxy resin, naphthalene-based epoxy resin, anthracene-based epoxy resin, bisphenol A diglycidyl ether epoxy resin, ethylene glycol diglycidyl ether epoxy resin, propylene glycol diglycidyl ether epoxy resin, or 1,4-butanediol diglycidyl ether epoxy resin.

12. The resin composition as claimed in claim 1, wherein the hardener is anhydride hardener, amine hardener, phenolic hardener, imidazole hardener, or a combination thereof.

13. A resin film, comprising a cured product of the resin composition as claimed in claim 1.
